(19) United States
(12) Patent Application Publication
(10) Pub. No.: US 2003/0171325 A1
(43) Pub. Date: Sep. 11, 2003

(54) Proofreading, error deletion, and ligation method for synthesis of high-fidelity polynucleotide sequences

(75) Inventors: Peter R.C. Gascoyne, Bellaire, TX (US); Daynene Vykoukal, Houston, TX (US); Frederick F. Becker, Houston, TX (US)

Correspondence Address: Michael C. Barrett, FULBRIGHT & JAWORSKI, L.L.P., Suite 2400, 600 Congress Avenue, Austin, TX, 78701, US

(73) Assignee: Board of Regents, The University of Texas System

(21) Appl. No.: 10/336,610

(22) Filed: Jan. 3, 2003

Related U.S. Application Data
(60) Provisional application No. 60/345,099, filed on Jan. 4, 2002.

(51) Int. Cl. A61K 31/70; A01N 43/04

(52) U.S. Cl. 514/44

(57) ABSTRACT

Methods and apparatuses for solid-phase oligonucleotide synthesis and forming long polynucleotides. One exemplary method includes synthesizing a sense oligonucleotide; synthesizing an antisense oligonucleotide; annealing the sense and antisense oligonucleotides to form double stranded DNA (dsDNA); capping the ends of the dsDNA; cleaving the dsDNA wherein cleavage occurs at or near a Watson-Crick base pair mismatch; and digesting uncapped dsDNA. Another exemplary method includes synthesizing a first proofread double stranded DNA (dsDNA); synthesizing a second proofread dsDNA; and ligating the first proofread DNA with the second proofread DNA to form a long polynucleotide.

### BACKGROUND OF THE INVENTION

This patent application claims priority to, and incorporates by reference, U.S. provisional patent application Serial No. 60/345,099 filed on Jan. 4, 2002 entitled, “Proofreading, Error Deletion, and Ligation Method for Synthesis of High-Fidelity Polynucleotide Sequences.”

I. Field of the Invention

The present invention relates generally to oligonucleotide synthesis. More particularly, it provides methods for proofreading oligonucleotide sequences, deleting errors, and methods for ligation. In different embodiments, these methods can be used with a microchip or in a microfluidic environment.

II. Description of Related Art

The ability to synthesize oligonucleotides and polynucleotides having a precise sequence is of fundamental importance to medical diagnostics, the life sciences, and the pharmaceutical industry. It is also important for environmental applications, including biological warfare detection. For example, such sequences may be used in the future as probes for known molecular signatures: In addition, if sufficiently long sequences are made, these can be used as synthetic genes and synthetic chromosomes to direct protein synthesis in living systems. Additionally, long nucleotide sequences may be used for information storage in devices such as molecular computers. Nature does not provide a mechanism for de novo synthesis of polynucleotides but always bases the structure upon an existing molecular template; therefore, in order to synthesize an arbitrarily specified sequence, some form of chemical synthesis must be employed.

One such method, called the phosphoramidite procedure, involves the systematic capping of reactive groups in the bases from which the polynucleotide is to be synthesized, followed by a sequence of reaction steps to unmask appropriate reactive sites and allow the reaction to form the desired polynucleotide. This method is able to accomplish step-wise accuracy in oligonucleotide sequences of 98.5%, a superb accomplishment for a chemical synthesis of a complex molecule. The yield of product having exactly the specified sequence in this method is (0.985)^{N}, where N is the number of nucleotide bases in the product. This allows oligonucleotide probes of 15-25 bases to be synthesized with reasonable purity. However, synthetic genes for chromosomes need sequences of anywhere from hundreds to tens of thousands of bases. At a 98.5% step-wise fidelity, a 10,000-base-long polynucleotide would be synthesized with a yield of accurate sequences of about 2×10⁻⁶⁴% by the phosphoramidite chemistry. This shows that chemical synthesis alone does not provide a viable mechanism for producing synthetic genes or chromosomes.

Techniques of the present disclosure overcome these disadvantages through the introduction of a new approach to the synthesis of high-fidelity sequences that provides for a step-wise fidelity of 99.9944%. This makes it feasible for the first time to synthesize artificial genes and chromosomes and also provides a superior method for making very high purity short oligonucleotide sequence for use as molecular probes etc. without the need for inefficient HPLC or other cleanup.

Any problems or shortcomings enumerated in the foregoing are not intended to be exhaustive but rather are among many that tend to impair the effectiveness of previously known processing and fluid injection techniques. Other noteworthy problems may also exist; however, those presented above should be sufficient to demonstrate that apparatus and methods appearing in the art have not been altogether satisfactory and that a need exists for the techniques disclosed herein.

### SUMMARY OF THE INVENTION

In one embodiment, the invention involves a method of solid-phase oligonucleotide synthesis. A sense oligonucleotide is synthesized. An antisense oligonucleotide is synthesized. The sense and antisense oligonucleotides are annealed to form double stranded DNA (dsDNA). The ends of the dsDNA are capped. The dsDNA is cleaved, wherein cleavage occurs at or near a Watson-Crick base pair mismatch, and uncapped dsDNA is digested.

In another embodiment, the invention involves a method of forming long polynucleotides. A first proofread double stranded DNA (dsDNA) is synthesized, wherein the synthesis includes: synthesizing a sense oligonucleotide; synthesizing an antisense oligonucleotide; annealing the sense and antisense oligonucleotides to form dsDNA; capping the ends of the dsDNA; cleaving the dsDNA, wherein cleaved dsDNA occurs at or near a Watson-Crick base pair mismatch; and digesting uncapped dsDNA. A second proofread dsDNA is synthesized. The first proofread DNA is ligated with the second proofread DNA to form a long polynucleotide.

In other respects, the invention includes apparatuses, systems, and/or software used to practice the methodology described herein.

Definitions

“Error” is defined herein as the error in the stepwise synthesis of a oligonucleotide. Error may be described as the percent of the time that a base added to the growing oligonucleotide chain is not the base that was intended to be added to the chain at that position. A synthesis with a high error has a low step-wise fidelity.

As used herein, the term “mismatch” is defined as a region of one or more unpaired or mispaired nucleotides in a double-stranded RNA/RNA, RNA/DNA or DNA/DNA molecule. This definition thus includes errors in the formation of an oligonucleotide and also includes mismatches due to insertion/deletion mutations and single and multiple base point mutations.

Nucleic acid sequences that are “complementary” are those that are capable of base-pairing according to the standard Watson-Crick complementarily rules. As used herein, the term “complementary sequences” means nucleic acid sequences that are complementary, or as defined as being capable of hybridizing to each other under stringent conditions such as those described herein. Similarly, the terms “sense” and “antisense” oligonucleotides refers to nucleic acid sequences that are complementary.

As used herein, a “carrier fluid” refers to matter that may be adapted to suspend other matter to form packets on a reaction surface. A carrier fluid may act by utilizing differences in hydrophobicity between a fluid and a packet. For instance, hydrocarbon molecules may serve as a carrier fluid for packets of aqueous solution because molecules of an aqueous solution introduced into a suspending hydrocarbon fluid will strongly tend to stay associated with one another. This phenomenon is referred to as a hydrophobic effect, and it allows for compartmentalization and easy transport of packets. A carrier fluid may also be a dielectric carrier liquid which is immiscible with sample solutions. Other suitable carrier fluid include, but are not limited to, air, aqueous solutions, organic solvents, oils, and hydrocarbons.

As used herein, a “programmable fluid processor” (PFP) refers to a device that may include an electrode array whose individual elements can be addressed with different electrical signals. The programmable fluid processor (PFP) can be configured to act as a programmable manifold that controls the dispensing and routing of reagents. As used herein, a “program manifold” is meant to describe the combination of computer controlled forces such as dielectric forces or magnetic forces, and systems which are used to control the movement of fluids and packets through a biochip.

As used herein, a “biochip” refers to a biological microchip which can be described as a nucleic acid biochip, a protein biochip, a lab chip, or a combination of these chips. The nucleic acid and protein biochips have biological material such as DNA, RNA or other proteins attached to the device surface which is usually glass, plastic or silicon. These biochips are commonly used to identify which genes in a cell are active at any given time and how they respond to changes. The lab chip uses microfluidics to do laboratory tests and procedures on a micro scale.

As used herein, an “oligonucleotide synthesis engine” (OSE) is a microfluidic device that exploits a wide range of effects that become dominant on the microfluidic scale including the hold-off properties of capillary tubes; the high pressures intrinsic to tiny droplets; the tendency of droplets to fuse and rapidly mix on contact with miscible solvents; the attractive and repulsive characteristics of surface energies for fluids in microfluidic spaces; and the ability of inhomogeneous AC electrical fields to actuate droplet injection and the trapping, repulsion and transport of dielectric particles. These effects can be used to realize a programmable fluid processor (PFP) based on the dielectrophoretic (DEP) injection and manipulation of droplets within an immiscible carrier fluid over a reaction surface consisting of a Teflon-coated, addressable electrode array.

As used herein, “packet” and “particle” both refer to any compartmentalized matter. The terms may refer to a fluid packet or particle, an encapsulated packet or particle, and/or a solid packet or particle. A fluid packet or particle refers to one or more packets or particles of liquids or gases. A fluid packet or particle may refer to a droplet or bubble of a liquid or gas. A fluid packet or particle may refer to a droplet of water, a droplet of reagent, a droplet of solvent, a droplet of solution, a droplet of sample, a particle or cell suspension, a droplet of an intermediate product, a droplet of a final reaction product, or a droplet of any material. An example of a fluid packet or particle is a droplet of aqueous solution suspended in oil. The packet or particle may be encapsulated or a solid. Examples of solid packets or particles are a latex microsphere with reagent bound to its surface suspended in an aqueous solution, a cell, a spore, a granule of starch, dust, sediment and others. Methods for producing or obtaining packets or particle as defined herein are known in the art. Packets or particles may vary greatly in size and shape, as is known in the art. In exemplary embodiments described herein, packets or particles may have a diameter between about 100 nm and about 1cm.

As used herein, an “array” refers to any grouping or arrangement. An array may be a linear arrangement of elements. It may also be a two dimensional grouping having columns and rows. Columns and rows need not be uniformly spaced or orthogonal. An array may also be any three dimensional arrangement.

As used herein, “a” or “an” may mean one or more. As used herein in the claim(s), when used in conjunction with the word “comprising,” the words “a” or “an” may mean one or more than one. As used herein “another” may mean at least a second or more.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a schematic drawing for a 4 mm×7 mm unit cell module. The module contains a programmable fluidic processor (PFP) that can be filled with non-polar partitioning medium, nucleotide and reagent droplets, a support bead reservoir and traveling wave dielectrophoresis (TWD) delivery system, accumulator and trapping electrode, a patterned surface with a wall-less flow path, a serial inlet and outlet, programmable fluidic processor dielectrophoresis (DEP) electrode array elements and reagent and rinse reservoirs with optional fluid bus inlets.

FIG. 2 is a plot showing the predicted behavior of engineered beads for five different microparticle types. Beads a, b, c, d and e are identical except for the thickness of the outermost, insulating shell which varies from 1 to 10 nm. Curves give the predicted DEP and TWD responses calculated from a Maxwell-Wagner dielectric dispersion associated with non-condicuting shells.

FIG. 3 is a plot showing the relationship between producing an oligonucleotide with no errors in sequencing and length of the oligonucleotide in number of bases.

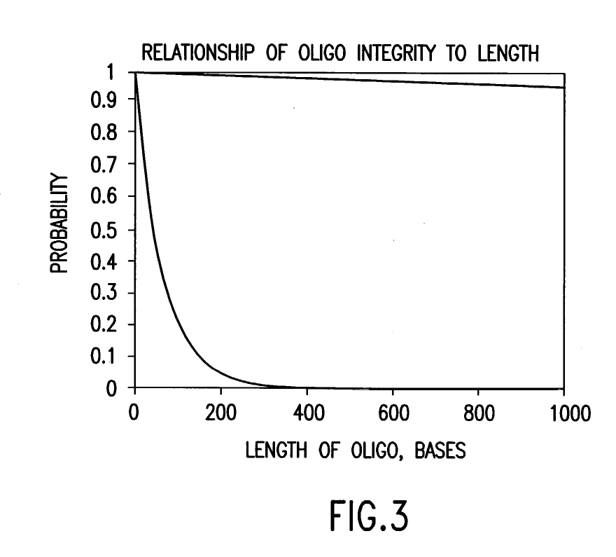

### DESCRIPTION OF ILLUSTRATIVE EMBODIMENTS

Techniques of the present disclosure overcome deficiencies in the art by providing a method for oligonucleotide synthesis with fewer errors than in current synthesis methods. It provides for, among other things, proofreading and error deletion in oligonucleotide synthesis. It also provides for, among other things, ligation methods for the synthesis of high fidelity nucleic acid products.

### I. OLIGONUCLEOTIDE SYNTHESIS

Based on the technology developed for solid-phase synthesis of polypeptides, the synthesis of nucleic acids with the initial nucleotide attached to a suitable solid support material has become possible. The use of a solid support aids in the automation of the synthesis process. Certain types of syntheses are now routinely carried out using automatic DNA synthesizers by sequentially adding activated monomers to a growing chain that is linked to an insoluble support. (Ike, Y., Ikuta, S., Sato, M., Huang, T., & Itakura, K. (1983) Solid phase synthesis of polynucleotides. Nucl. Acid. Res., 11, 477, the entirety of which is herein incorporated by reference).

Synthesis of a specific oligonucleotide sequence may be done using a programmed series of reagent additions to accomplish the extension, washing and deprotection steps as the product is extended. A conventional approach to this problem demands numerous valves and tubes and other fluid handling components that, in turn, demand an enormously complex micromechanical system, which would be prone to mechanical failure if reduced to chip-scale. The ability to move droplets along arbitrarily chosen and crossing paths on a two dimensional reaction surface eliminates the need for tubes and vials required in microfluidic adaptations of conventional channel-based fluidic designs. The use of a dielectrophoresis (DEP) based programmable fluidic processor (PFP) allows for reconfigurable, channel-less fluid handling, enabling programmed, multiplexed, and/or parallel microfluidic protocols to be executed. This approach eliminates the need for microfluidic valves, mixers, and explicit metering, and it overcomes carryover and dead-space issues.

A successful oligonucleotide synthesis system should be able to generate high quality oligonucleotides, use minimal amount of reagents and solvents, and have a very short cycle time for stepwise reactions. The determination of appropriate protocols may involve: (a) development of chemistry for derivatization of dielectrically-engineered microbead surfaces with linkers and functional groups suitable for oligonucleotide synthesis; (b) optimization of solvent and reagent systems for oligonucleotide synthesis using DEP-driven delivery; (c) development of methods to monitor oligo synthesis and characterize the final products. One suitable approach is based on the nucleo-phosphoramidite chemistry using trichloroacetic acid (TCA) or other organic acid as the deprotecting agent and, thus far, is the most efficient way to achieve high yield synthesis of oligonucletodies. Other chemistries may also be used, as will be understood by those having skill in the art, following the protocols known in the art.

a. Phosphoramidite Chemistry

The phosphoramidite chemistry can be optimized, for example, by using different solvents for improved dielectrophoretic transport, surface wettability, and/or volatility. Reaction mechanisms in oligonucleotide chemistry are well-characterized. Further, studies in developing synthesis protocols using phosphoramidite chemistry, adaptation of reaction parameters, including chemical stochoimetry, reaction times, solution volumes, and solvents are efficient and relatively rapid. Examples of alternative solvent systems include, but are not limited to, detritylation in propylene carbonate or toluene, and varying the ratio of THF:pyridine in the capping reaction.

Phosphoramidite chemistry (Beaucage et al., 1992; EP 266,032, which is incorporated herein by reference) involves activation of nucleoside phosphoramidite monomer precursors. The activated monomers are protonated deoxyribonucleoside 3′-phosphoramidites. First, the 3′-phosphorus atom of the phosphoramidite joins to the 5′-oxygen of the growing chain to form a phosphite triester. The 5′-OH of the activated monomer is unreactive because it is blocked by a dimethoxytrityl (DMT) or other protecting group. Coupling is preferably carried out under anhydrous conditions because water reacts with phosphoramidites. In the second step, the phosphite triester is oxidized by iodine to from a phosphotriester (the phosphorus goes from trivalent to pentavalent). Next, the DMT protecting group on the 5′-OH of the growing chain is removed by addition of TCA, dichloroacetic acid, or another organic acid which leaves any other protecting groups intact. The oligonucleotide chain has then been elongated by one base and is ready for another cycle of addition. (Stryer, L “Biochemistry”, Freeman and Co., 1995, which is incorporated herein by reference). Examples of oligonucleotide synthesis using solution photogenerated acids which are suitable for removal of the acid labile protection group on 5′-O of nucleotides have been described in the art. (Gao et al., 1998; Pellois et al., 2000, which are incorporated herein by reference).

The phosphoramidite method, employing nucleotides modified with various protecting groups, is the most commonly used method for the de novo synthesis of polynucleotides. Its reaction efficiency is good for a chemical synthesis scheme and is well suited for the generation of short oligonucleotide probes and primers. The error rate of phosphoramidite oligonucleotide synthesis has been shown to provide a 98.5% stepwise fidelity. This translates to fidelity for a sequence of N bases of (0.985)^{N}.

In one embodiment of this disclosure, a chip-scale implementation of this method using DEP reagent handling on PFP may be used. This stepwise fidelity, however, may be highly problematic for synthesizing long polynucleotides because the yield of accurate sequences falls exponentially with sequence length. Living systems contain various enzymatic-proofreading mechanisms for identifying errors in DNA. Several of these have been characterized and adapted for detecting point-mutations in patient samples. These enzymatic methods, as well as established chemical cleavage methods, may be used so that error-containing polynucleotide sequences are identified, cleaved and eliminated by nuclease digestion, leaving the correctly synthesized sequence intact.

b. Solid Support

The use of a solid phase approach is advantageous for oligonucleotide synthesis at least because the desired product stays on the insoluble support until the final release step. All reactions may occur in a single vessel or on a single chip where excess soluble reagents can be added to drive reactions to completion. At the end of each step, soluble reagents and by-products may be washed away from beads that bear the growing chains. At the end of the synthesis, NH₃ may be added to remove all protecting groups and release the oligonucleotide from the solid support.

The solid-phase support may used to retain oligonucleotides after synthesis. Recognizing that the attachment to surfaces of a PFP may compromise a degree of on-the-fly reconfigurability and reusability that may be desirable, novel microspheres can be used as mobile solid support for oligo synthesis according to one embodiment of this disclosure. The fabrication methods for beads can be modified to provide appropriate microspheres for mixed-solvent systems and to develop a traveling-wave DEP delivery-on-demand system for metering, injection and transport of the beads. The ability to reversibly immobilize oligonucleotides in a microfluidic device under electrical control without having to link them directly to the surface of the device as taught herein represents a major advance in microflume-based molecular analysis and synthesis.

Dielectrically-engineered beads with well-controlled dielectric properties may serve as the solid phase anchors for oligo synthesis. In one embodiment, these beads allow attached oligos to be transported by traveling-wave DEP, trapped by positive DEP against fluid flow during rinsing, stirred by alternate DEP trapping and repulsion, released and flushed from the PFP into receiving stages for further processing after completion of oligonucleotide synthesis, and generally manipulated by DEP. The microspheres can be trapped by positive DEP and repelled by negative DEP by changing the frequency of the applied DEP filed. The microspheres can be fabricated for single and mixed-solvent systems. These microspheres can be metered, infected and transported to the PFP using traveling wave DEP, pressure, and differing surface energies (Wang et al., 1997, 1992, which are incorporated herein by reference).

Beads may be designed to mimic the dielectric structure of a mammalian cell and may contain a highly conductive core surrounded by a thin, electrically insulating membrane. These microspheres undergo a frequency-dependent change in AC conductivity and can be trapped by positive DEP or repelled by negative DEP by changing the frequency of the applied field. Without being bound by theory, it is believed that this behavior results from a Maxwell-Wagner dielectric dispersion associated with non-conducting shell.

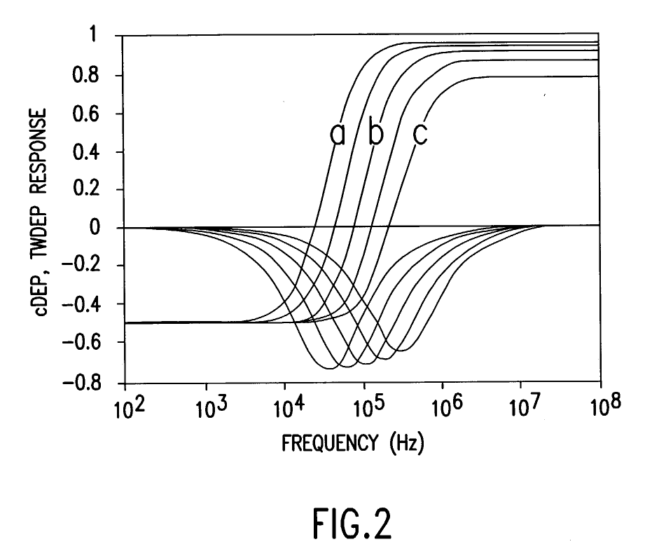

FIG. 2 illustrates the calculated DEP and TWD responses for five different microparticle types. Each bead is identical except for the thickness of the outermost, insulating shell, which varies from 1-10 nm.

The surface of the beads may be modified to accommodate the chemical requirement of organic synthesis. In a nonlimiting example, the inventors have fabricated engineered microspheres by forming self-assembled insulting monolayers (SAMs) of alkanethiolate and phospholipid on gold-coated polystyrene core particles. Alkanethols CH₃(CH₂)_{η}—SH, chemisorb spontaneously onto gold surfaces to form alkanethiolates that self-organize into densely packed, robust monolayer films (Wasserman et al., 1989, which is incorporated herein by reference). An additional, self-assembled monolayer film of phospholipid can be applied over the alkanethiolate SAM to increase the thickness of the engineered microsphere and yield a polar, hydrophilic outer surface. One bead design that has been shown to be useful consists of gold-coated polystyrene core particles of uniform size (10 microns diameter) that have been coated with self-assembled monolayers of alkane thiol and subsequently converted to a hybrid bilayer membrane by an additional self-assembled phospholipid monolayer coating step that is able to produce a stable, cross-linked polymeric coat of precisely defined thickness.

The effects of spacers, linkage and solid support on the synthesis of oligonucleotides may be utilized and has been described by Katzhendler et al., (1989) while other methods for synthesis on a surface have been described, for example, by LeProust et al. (2000, 2001). The bead design can be adapted for use as oligonucleotide anchors, for example by the attachment of thiolated oligonucleotide primer sequences or by adding various coatings that allow the attachment of other types of linkers for chemical synthesis, such as polyethyleneglycol terminated with a hydroxyl or silicon based materials.

The ability to reversibly immobilize oligonucleotides in a microfluidic device under electrical control without having to link them directly to the surface of the device is a major advance in microflume-based molecular analysis and synthesis. Beads allow the reversible immobilization and transport of oligos under DEP (or any other electronic control) and obviate the need for direct interactions of oligos with the surface of the chip. To allow multiple, sequential syntheses, a bead reservoir and a bead dispenser may be used.

In different embodiments, the surface of the solid support may include, for example, polystyrene, phospholipid, polyethylene glycol, controlled pore glass or a derivatized membrane. The solid support may include a surface layer that has been designed to bind to the nucleic acid bases for oligonucleotide synthesis and an interior that has been designed to be manipulated by external forces such as DEP. Preferably, the solid support can be manipulated by a dielectric field.

It will be understood that numerous other materials may be used in the solid support, including, but not limited to, nitrocellulose, nylon membrane, glass, reinforced nitrocellulose membrane, activated quartz, activated glass, polyvinylidene difluoride (PVDF) membrane, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules (Saiki, et al., 1994, which is incorporated herein by reference)) and magnetic controlled pore glass described in U.S. Pat. No. 5,601,979, which is hereby incorporated by reference.

### II. PROOFREADING AND ERROR DETECTION

De novo synthesis of oligos by chemical methods such as the phosphoramidite approach results in products with stepwise fidelity of 98.5%. While this is a superb accomplishment for a complex organic synthesis and is routinely used for making probes and primers 15 to 25 bases long, it is unacceptable for polynucleotides longer than 30 or so bases. Because of the fundamental importance of accurately conserving the genome, nature not only relies on template-mediated synthesis but also incorporates highly evolved enzymatic error detection and correction mechanisms. Although the present application involves synthesis proceeding de novo, it is nevertheless possible to harness proofreading machinery to greatly improve the fidelity of synthetic polynucleotides. In this vein, an entirely new proofreading and error elimination system may be provided to enhance fidelity of de novo polynucleotide synthesis.

In order to greatly enhance synthesis fidelity for long nucleotide sequences, a novel proofreading system to enhance fidelity of de novo polynucleotide synthesis that eliminates error-containing sequences is herein disclosed. In one embodiment, desired oligo sequences and their complimentary sequences are independently synthesized. The complimentary sequences are paired, and base-pair mismatches are detected. This aqueous method may be based on the exploitation of known enzymatic and chemical cleavage of DNA containing mismatched bases using Watson-Crick base pairing, followed by subsequent chemical or enzymatic digestion of cleaved sequences by an appropriate nuclease. This method eliminates errors except double-errors that result in fortuitous Watson-Crick pairing between the complimentary strands. This strategy reduces the stepwise error rate by almost three orders of magnitude compared with the error rate for chemical synthesis. The remaining intact sequences should have a 99.9944% stepwise fidelity. The final product yield after digestion should be (0.985)^{2N}. For example, if 10⁷ sense and antisense oligos of length 100 bases were synthesized, the yield after proofreading and digestion steps would be approximately 5×10⁵ DNA molecules. Of these, 99.9944% will be free of fortuitous compensatory errors.

The use of Watson-Crick base pairing to detect differences in oligonucleotides has been described previously (Meyer et al., 2001; Barany et al., 1991; Wu et al., 1989, each of which is incorporated herein by reference). Meyer et al., describe a PCR-based approach for the synthesis of ligation probes. When hybridized to a target, the probes form a nicked circle that may be sealed by DNA ligase only if the 5′ and 3′ ends show perfect Watson-Crick base pairing. This allows for the detection of SNPs and any other discrepancies between the two oligonucleotides.

Multiple cleavage techniques have been developed to exploit this structural change by selectively degrading or modifying DNA at the site of the error. Ideally, little or no cleavage is seen in a perfectly matched DNA fragment, and all distortions of the helix generated by base mismatches result in cleavage. In practice, neither criteria are fully met, and the utility of a technique becomes a trade-off between ease of use, sensitivity and specificity (Taylor et al., 1999).

a. Chemical Cleavage

Methods for cleavage of errors in the oligonucleotide sequences may be based upon the interaction of chemical moieties and the oligoncleotides.

Chemical Cleavage of Mismatch

Chemical cleavage of mismatch (CCM), also known as chemical mismatch cleavage (CMC) or the HOT (hydroxylamine/osmium tetroxide) chemical method is one technique for detecting and localizing mismatches in DNA molecules which was originally described by Cotton (Cotton et al. 1988; Lambrinako et al., 1999). CCM is described in detail by Saleeba, et al. (1993) and by Ellis et al. (1998). Potassium permanganate can be used in place of the osmium tetroxide (Roberts et al., 1997).

CCM relies upon the chemical reactivity of mismatched C and T bases to hydroxylamine and osmium tetroxide, respectively. Once reacted, the DNA strands are cleaved at the reacted mismatched base by piperidine and the molecules are separated by size to identify the location of the mismatched positions. This method is highly sensitive, with a sensitivity approaching 100%.

U.S. Pat. No. 5,972,618, which is incorporated herein by reference, describes a CCM wherein a piece of control nucleic acid is annealed without mutations to a piece of test nucleic acid very similar in sequence to the control nucleic acid but possibly containing mutations, treating this mixture with potassium permanganate or hydroxylamine to remove mismatched bases from the duplex nucleic acid, treating the resulting nucleic acid with an analogue to 1,2-ethylenediamine to cleave abasic sites, and then analyzing the chemically treated nucleic acid to determine whether cleavage has occurred and approximately at what position in the nucleic acid any cleavage has occurred.

One example of a CCM is described by the following steps: 1) PCR of normal DNA with two fluorescent primers and mutant DNA with two biotinylated primers or fluorescent nucleotides and mutant DNA with fluorescent nucleotides and two biotinylated primers; 2) denature and anneal PCR products in annealing buffer; 3) add streptavidin-magnetic beads and hydroxylamine or potassium permanganate to product; 4) incubate for 2 hours at 37° C. or 1 hour at 25° C.; 5) remove supernatant and re-suspend beads; 6) incubate at 90° C. 30 minutes; and 7) snap chill and load on a denaturing gel or a DNA sequencer.

Other examples of CCM technique can be found by, for example, Axton et al. (1997) where PAX6 mutation are detected; Draghia et al., (1997) where the first deletion in exon 1 and of nine novel point mutations are found; and Germain et al. (1996) where fluorescence-assisted mismatch analysis is used to screen the alpha-galactosidase. The method is very robust and semi-automatable since modifications have been introduced including fluorescent detection and solid-phase capture of the heteroduplex (Rowley et al., 1995).

Rhodium Intercalator

Photoactivated rhodium DNA intercalators have been used for mismatch detection. Rhodium(III) complexes initiate photoactivated cleavage (Jackson et al., 1997; Jackson et al., 1999). For cleavage by Rh(DIP)₃³⁺, the photoactived complex has been shown to target specifically guanine-uracil (G-U) mismatches (Chow et al., 1992). Other rhodium DNA intercalators (such as [Rh(bpy)₂(chrysi)]³⁺) are both a general and remarkably specific mismatch recognition agent having specific DNA cleavage at over 80% of mismatch sites in all the possible single base pair sequence contexts around the mispaired bases (Jackson et al., 1999). Other rhodium(III) complexes bind and, with photoactivation, cleave DNA at increased reactivity Kisko et al., (2000).

b. Enzymatic Mismatch Cleavage

Enzymatic methods for determining mismatch may also be used in the proofreading and deletion methods of embodiments of the current disclosure. Multiple enzymetic methods have been developed. Developments in the understanding of enzymatic mismatch recognition process has improved the sensitivity and specificity of these methods, and several enzymatic methods such as those described by Tayler et al. (1999) may be used in error detection in embodiments herein.

DNA Endonucleases

T4 endonucleases such as T4 endonuclease V, T4 endonuclease VII (T4E7) and T7E1 are small proteins from bacteriophages that bind as homodimers and cleave aberrant DNA structures including Holliday Junctions (and are hence sometimes called “resolvases”) though it is far from clear that they perform such a role in vivo (Pohler et al., 1996). Others (Youil et al., 1995; Mashal et al., 1995; White et al., 1997) observed that they preferentially cleave mismatched heteroduplexes, leading to the possibility of an enzymatic equivalent to the chemical cleavage of mismatch. Distinct hemoglobin mutations have been detected (Youil et al., 1996). At present there is a somewhat higher background than seen with chemical cleavage although this is clearly an approach that has great potential and may constitute a suitable method. DNA requires no special preparation after amplification like GC clamping or including primers with ′phage promoters. Background peaks which are seen are highly reproducible and may therefore amenable to background subtraction algorithms like those applied to DNA sequencing traces (Bonfield et al., 1998).

T4 endonuclease V initiates the process of repairing UV-damaged DNA by catalyzing the excision from either strand of DNA of pyrimidine dimers formed as a result of irradiation (Yao et al., 1997). In vivo and under low salt conditions in vitro, the enzyme binds to the DNA through electrostatic forces, then diffuses along the DNA by a sliding mechanism until it reaches its target site: a pyrimidine dimer (Gordon et al., 1980; Lloyd et al., 1980). A commercially available source of T4 endonuclease V is available from Worthington and Trevigen, and a T4E7-based mutation detection kit is available from Amersham-Pharmacia. A plant endonuclease (CEL I) with similar activity has also been described (Oleykowski et al., 1988). CEL I is one of series of plant endonucleases with similar activity to nuclease S1but at neutral pH instead of pH 4 or 5. Like T4E7, the cleavage efficiency varies according to the mismatch examined and background cleavage is dependent on the template being examined.

EMC

Enzymatic Cleavage of Mismatch (EMC) is another suitable method, including an improved Enzymatic Mutation Detection (EMD™) assay. EMD is a fully homogeneous, rapid four step procedure that allows for detection and localization of mismatched or unmatched nucleotides within heteroduplex DNA. These are sensitive and rapid methods for to mutation detection in large genes.(Youil, 2000)

Ribonuclease A

A method for screening for point mutations is based on RNase cleavage of base pair mismatches in RNA/DNA and RNA/RNA heteroduplexes. Currently available RNase mismatch cleavage assays, including those performed according to U.S. Pat. No. 4,946,773, which is incorporated herein by reference, require the use of radiolabeled RNA probes. Myers and Maniatis in U.S. Pat. No. 4,946,773 describe the detection of base pair mismatches using RNase A. Other investigators have described the use of an E. coli enzyme, RNase I, in mismatch assays. Because it has broader cleavage specificity than RNase A, RNase I may be a desirable enzyme to employ in the detection of base pair mismatches if components can be found to decrease the extent of non-specific cleavage and increase the frequency of cleavage of mismatches.

The RNase protection assay was first used to detect and map the ends of specific mRNA targets in solution. The assay relies on being able to easily generate high specific activity radiolabeled RNA probes complementary to the mRNA of interest by in vitro transcription. Originally, the templates for in vitro transcription were recombinant plasmids containing bacteriophage promoters. The probes are mixed with total cellular RNA samples to permit hybridization to their complementary targets, and the mixture is then treated with RNase to degrade excess unhybridized probe. The RNase Protection assay was adapted for detection of single base mutations. In this type of RNase A mismatch cleavage assay, radiolabeled RNA probes transcribed in vitro from wild-type sequences, are hybridized to complementary target regions derived from test samples. The test target generally comprises DNA (either genomic DNA or DNA amplified by cloning in plasmids or by PCR™), although RNA targets (endogenous mRNA) have occasionally been used.

If single nucleotide (or greater) sequence differences occur between the hybridized probe and target, the resulting disruption in Watson-Crick hydrogen bonding at that position (“mismatch”) can be recognized and cleaved in some cases by single-strand specific ribonuclease. To date, RNase A has been used almost exclusively for cleavage of single-base mismatches, although RNase I has recently been shown as useful also for mismatch cleavage.

Mismatches have been detected by means of enzymes such as RNaseA, which cut one or both strands of the duplex at the site of a mismatch. Duplexes without mismatches are not cut. By using radioactively labeled nucleic acid fragments to anneal to a test DNA, it is possible to use these enzymes to generate specific size fragments when a mutation is present in the test DNA. The fragments are distinguished from uncut fragments by means of polyacrylamide gel electrophoresis. Ribonuclease A cleavage was originally described by Myers et al. (1985) using DNA:RNA hybrids. Sensitivity was reported to be around 60% per strand cleaved. Grange et aL(1990) described improved sensitivity by screening both strands of RNA. A Non-Isotopic RNase Cleavage Assay (NIRCA) has also been described (Goldrick et al., 1996). As a fast and easy screening method for large fragments, this method has much in its favor (Gibbons et al., 1997). The major disadvantages are lack of 100% sensitivity and the need to make primers which include 'phage polymerase promoters. However since large (1 Kbp) DNA fragments are often amplified with 'phage promoters as part of the protein truncation test, it may be cost-effective to use those amplifiers to produce template for RNase cleavage

MutY and Thymine Glycosylase

MutY acts similarly to RNaseA and has considerable potential for mismatch detection, as its in vivo function is to repair mismatched G:A base pairing by cleavage of the adenine-containing strand. Similar proteins thought to be involved in G:T and G:U mismatch repair have also been described (Neddermann et al, 1996). Lu and Hsu (1992) described the use of E. coli MutY protein for the detection of mismatched G:A in p53. A major limitation was that only G:A mispairs were detected. Hsu et al.(1994) described the use of MutY in combination with thymine glycosylase for mismatch detection. In this method, DNA fragments amplified from normal and mutated genes by polymerase chain reaction (PCR) were mixed and annealed to create DNA mismatches for cleavage by mismatch repair enzymes. The cleaved products and the substrates were separated by gel electrophoresis and detected by autoradiography. All mutated DNA samples yielded cleaved products with sizes as expected with low background. As described, the method offers no way of detecting G:G or C:C mismatches.

MBP

Immobilized mismatch binding protein (MBP) such as the MutS protein of E. coli has been used for the detection of genetic mutations or genomic polymorphisms and the purification of DNA samples by removing contaminating sequences and sequences containing errors (U.S. Pat. Nos. 6,114,115 and 6,027,877, both of which are incorporated herein by reference).

MutS and homologues are mismatch recognition proteins originally identified in “mutator” strains of E. coli. Lahue et al., (1989) have completely reconstructed MutS initiated mismatch repair in vitro. The E. coli MutS protein recognizes single base mismatches (with the exception of C:C mismatches). Eukaryotic MutS homologue binding is via an heterodimer of hMSH2 and either hMSH3 (MutS alpha) or hMSH6 (MutS beta) in humans (Modrich et al., 1997). There are reports that hMSH2 can bind mismatches in the absence of hMSH3 or 6 (Fisher et al., 1997). The mismatch binding of MutS has been exploited for mutation detection in several formats; solid-phase capture of mismatched heteroduplexes, mobility-shift assays. Two cleavage assays using MutS have been described: mismatch protection from exonuclease (MutEx), which also enables the mutation to be localised (Ellis et al., 1994) and by utilization of an in vitro reconstructed MutHLS system (Smith et al, 1996). The MutEx assay works well on a subset of mismatches, giving clear peaks and almost no background, but MutS binding to other mismatches is not always strong enough to prevent exonuclease reading through the site of the mismatch. Cross linking of MutS to the mismatch may alleviate this problem. The MutEx assay has been used increase the fidelity of PCR products by removing artifacts caused by polymerase errors (Smith et al., 1997).

Uracil Glycosylase and BESST and G Scans

Uracil glycosylase and proprietary (Epicentre) photo-activated guanine modification reagent have been used to develop a cleavage method that essentially produces T or G sequencing tracks. DNA synthesis by PCR requires the incorporation of a proportion of uracil bases in place of thymines. These can be removed by uracil glycosylase and the abasic site then cleaved by heat or enzymatic treatment. The resulting digest is then resolved on a sequencing gel to reveal the positions of the T bases (Hawkins et al., 1997). A similar approach using photochemical modification and cleavage of G bases may detect all point mutations.

### III. LIGATION

If the phosphoramidite synthesis scheme were used to create a 10,000 base long polynucleotide, there would be a yield of only 2×10⁻⁶⁶. In order to reduce this error rate to a level that makes feasible the production of long polynucleotides at high yield, the current disclosure introduces an innovative scheme coupling proofreading and error elimination with ligation.

The error deletion method of the current disclosure may be combined with ligation to produce oligomers with lengths greater than can be effectively produced by the direct synthesis of oligomers. Short DNA sequences can be ligated into synthetic chromosomes. Following proofreading and elimination of erroneous sequences, multiple short, double-stranded DNA subunits can be ligated to yield long, high fidelity, synthetic chromosomes. In one embodiment, this ligation can occur in a PFP and may be under automated, electronic control.

After independently synthesizing short complimentary oligos, the antisense sequence may be cleaved from the bead support and annealed with the sense sequence which is still attached to beads to provide dsDNA. Chemical cleavage of mismatch (CCM) chemistry or enzymatic cleavage of mismatch (ECM) methods may then be used to cleave error-containing DNA, rendering it susceptible to enzymatic digestion by appropriate nucleases. In principle, the only remaining errors in the DNA will result from fortuitous compensatory errors that maintain Watson-Crick base pairing in the complimentary strands. Since the optimized stepwise fidelity for single-strand phosphoramidite synthesis is 0.985, the stepwise probability of such compensatory errors in aired complimentary strands is [1−0.25*(1−0.985)2]N, or about 1 error per 18,000. The proportion yield of accurate DNA is (0.985)^{2N}, where N is the length of the polynucleotide.

The on-chip ligation of proofread sequences prepared using this scheme allows for the synthesis of 10,000 base pair synthetic chromosomes with greater than 50% perfect sequence yield. Embodiments of this disclosure include a chip-scale, microfluidic oligonucleotide synthesizer based on a CMOS version of a PFP that realizes this high fidelity synthesis and proofreading scheme. The design may be scaleable and suitable for batch fabrication in modular or integrated form. Thus, the synthesis engine may work not only for short-chain oligonucleotide synthesizers suitable for integration into microscale diagnostic and chem-bio and/or warfare agent detection systems, but also for massively integrated, long-chain polynucleotide synthesis applications.

Synthesis of long DNA sequences may be accomplished by the ligation of multiple, separately synthesized and proofread subunits of double-stranded DNA of appropriate sequences. Ligase is often obtained from E. coli, which has been infected with the T4 bacteriophage. This requires ATP as a co-factor, but the T4 ligase produced has the ability to join blunt ends well. Other ligase methods known in the art may also be used. Ligation occurs when enzyme or chemical activity allows the joining between ends of DNA segments. Ligation can be used in the present disclosure to prepare strands of DNA where the final DNA has a length greater than can be obtained efficiently solely using step-wise synthesis.

When the final product is a long single strand, this fabrication approach has the advantage of eliminating difficulties of tangling and formation of secondary structures that are associated with long single-stranded polynucleotide syntheses. When single-stranded sequences are desired, they can be made by nuclease digestion of the redundant strand from double-stranded DNA.

The enzyme T4 DNA ligase is commonly used for carrying out ligation reactions. T4 works best on cohesive ends of DNA, but it will also knit together blunt ended DNA if the DNA is in high enough concentration and if enough enzyme is added. In either case, the molecules to be joined must have a phosphate group at its 5′ end, and a hydroxyl at its 3′. There are three fundamental ways of joining DNA molecules together: cohesive end ligation, complementary homopolymer ligation, and blunt end ligation, each of which may be used in embodiments of the current disclosure.

a. Cohesive End Ligation

Cohesive ends are complementary single stranded regions found at the ends of DNA molecules. As mentioned above, many, but not all, restriction enzymes form single-stranded ends of this nature when they cut DNA. For example, when the restriction endonuclease EcoRI cleaves the sequence:

It leaves the ends:

5′ ....GAATTC.... 3′
3′ ....CTTAAG.... 5′
5′ ....G 3′ 5′ AATTC.... 3′
3′ ....CTTAA 5′ 3′G.... 5′

Under the appropriate conditions, in the presence of either E. coli or T4 DNA ligase, these ends can be joined together to reform a complete molecule. In particular, if a circular DNA molecule (such as a plasmid) is cleaved once with EcoRi, a linear molecule with two cohesive ends results. A second piece of foreign DNA may be inserted into these sites, reforming a circle if it has the same ends.

There are at least two competing reactions that can occur: reformation of the original circular DNA and circularization of the foreign DNA. Reaction conditions are usually set up to avoid these side-reactions. Circularization of the vector is favored at low DNA concentrations because its two complementary ends are always in the same vicinity (e.g. part of the same molecule). As the concentration of the foreign DNA is increased, reactions between separate molecules will be more frequent than ones within the same molecule.

b. dA/dT and dC/dG Joining

Two oligonucleotide fragments can be joined together by the addition of complementary homopolymers to the 3′ ends of two fragments of DNA using the mammalian-derived enzyme terminal transferase. When presented with deoxynucleotide triphosphates, this enzyme will add nucleotides to the 3′ OH ends of a DNA molecule. For example, if poly dT is added to the 3′ ends of one fragment, and poly dA to the 3′ ends of another, the two fragments can join together.

c. Blunt End Ligation

Two oligonucleotide strands with no regions of single-stranded complementary can also be joined, provided that their 5′ ends have terminal phosphate groups and their 3′ hydroxyl groups are not blocked. T4 ligase has this blunt end ligating activity. Another application of the blunt end ligation activity of T4 ligase is to join synthetic DNA “linkers” on to DNA fragments. Linkers are short, symmetrical, self-complementary oligonucleotides that have one or more restriction sites within them.

d. DNA Size

Following proofreading and elimination of erroneous sequences, multiple short, double-stranded DNA subunits can be ligated in a PFP to yield long, high fidelity, synthetic chromosomes. This method may be done rapidly in an automated system on the microscale using a PFP as described herein. The synthetic oligonucleotide may be short, comprising up to 100, 200, 300, 400, 500, 600, 700, 800 or 900, or it be long, comprising up to 1,000, 2,000, 3000, 4000, 5,000, 6,000, 7000, 8000, 9000, 10,000, 11,000, 12,000, 13,000, 14,000, 15,000, 16,000, 17,000, 18,000, 19,000, 20,000, 25,000, 30,000, 35,000, 40,000, 50,000, 60,000, 70,000, 80,000, or more base-pair in length.

The choice of the initial oligo length, N, for the short DNA sequence and of the number of serial versus parallel synthesis processes to used to make the desired synthetic chromosome may be determined, for example, by the desired speed, the required yield, and the allowable complexity for the compete synthesizer system. The length N of the short DNA may be up to 10, 20, 30, 40, 50, 60, 70, 80, 90, 100, 110, 120, 130, 140, 150 or more base-pair in length. There may be up to 10, 20, 30, 40, 50, 60, 70, 80, 90, 100, 110, 120, 130, 140, 150 or more short DNA sequences made in parallel. These oligonucleotides can then be combined to make the desired synthetic chromosome.

### IV. DIELECTROPHORETIC FLUIDIC SYSTEMS

Technology in fluidic and microfluidic systems has advanced such that there are numerous devices and systems available for the synthesis, manipulation, and analysis of small chemical and biological samples. These devices allow for rapid and automated synthesis which can be done on demand, without the need for storage of a nucleic acid product between synthesis and use. This disclosure provides technologies for the automated, chip-scale synthesis of oligonucleotide sequences of high purity. Aspects of embodiments herein couple the development of unique integrated microfluidic handling methods with high purity oligonucleotide synthesis techniques. One core microfluidic “deliverable” of this disclosure is a functional oligo synthesis module specifically designed for integration into other instruments, including instruments such as those described below.

The proofreading methods of the current disclosure can be used with, for example, the apparatus described in U.S. Pat. No. 6,294,063, entitled, “Method And Apparatus for Programmable Fluidic Processing,” which is incorporated herein by reference in its entirety. This patent discloses techniques that relate to the manipulation of a packet of material using a reaction surface, an inlet port, means for generating a programmable manipulation force, a position sensor, and a controller. In one embodiment of that disclosure, a material is introduced onto the reaction surface with the inlet port. The material is compartmentalized to form a packet. The position of the packet is tracked with the position sensor. A programmable manipulation force (which, in one embodiment, may involve a dielectrophoretic force) is applied to the packet at a certain position with the means for generating a programmable manipulation force, which is adjustable according to the position of the packet by the controller. The packet may be programmably moved according to the programmable manipulation force along arbitrarily chosen paths.

Other patents and applications that may be used in conjunction with techniques of the current invention include U.S. Pat. No. 5,858,192, entitled “Method and apparatus for manipulation using spiral electrodes,” filed Oct. 18, 1996 and issued Jan. 12, 1999; U.S. Pat. No. 5,888,370 entitled “Method and apparatus for fractionation using generalized dielectrophoresis and field flow fractionation,” filed Feb. 23, 1996 and issued Mar. 30, 1999; U.S. Pat. No. 5,993,630 entitled “Method and apparatus for fractionation using conventional dielectrophoresis and field flow fractionation,” filed Jan. 31, 1996 and issued Nov. 30, 1999; U.S. Pat. No. 5,993,632 entitled “Method and apparatus for fractionation using generalized dielectrophoresis and field flow fractionation,” filed Feb. 1, 1999 and issued Nov. 30, 1999; U.S. patent application Ser. No. 09/395,890 entitled “Method and apparatus for fractionation using generalized dielectrophoresis and field flow fractionation,” filed Sep. 14, 1999; U.S. patent application Ser. No. 09/883,109 entitled “Apparatus and method for fluid injection,” filed Jun. 14, 2001; U.S. patent application Ser. No. 09/882,805 entitled “Method and apparatus for combined magnetophoretic and dielectrophoretic manipulation of analyte mixtures,” filed Jun. 14, 2001; U.S. patent application Ser. No. 09/883,112 entitled “Dielectrically-engineered microparticles,” filed Jun. 14, 2001; U.S. patent application Ser. No. 09/883,110 entitled “Systems and methods for cell subpopulation analysis,” filed Jun. 14, 2001; U.S. patent application Ser. No. 10/005,373 entitled “Particle Impedance Sensor,” by Gascoyne et al., filed Dec. 3, 2001; U.S. patent application Ser. No. 10/028,945 entitled “Dielectric Gate and Methods for Fluid Injection and Control” by Gascoyne et al., filed Dec. 20, 2001; U.S. patent application Ser. No. 10/027,782 entitled “Forming and Modifying Dielectrically-Engineered Microparticles” by Gascoyne et al., filed Dec. 20, 2001; U.S. patent application Ser. No. ______ entitled “Droplet-Based Microfluidic Oligonucleotide Synthesis Engine” by Gascoyne et al., filed Jan. 3, 2003; and U.S. patent application Ser. No. ______ entitled “Wall-less Channels for Fluidic Routing and Confinement” by Gascoyne et al., filed Jan. 3, 2003; each of which is herein incorporated by reference.

Yet another application that may be used in conjunction with the teachings of the current invention include those described in “Micromachined impedance spectroscopy flow cytometer of cell analysis and particle sizing,” Lab on a Chip, vol. 1, pp. 76-82 (2001), which is incorporated by reference.

a. Programmable Fluidic Processor

A programmable fluid processor (PFP) may include an electrode array whose individual elements can be addressed with different electrical signals. The addressing of electrode elements with electrical signals may initiate different field distributions and generate dielectrophoretic or other manipulation forces that trap, repel, transport, or perform other manipulations upon packets of material on and above the electrode plane. By programmably addressing electrode elements within the array with electrical signals, electric field distributions and manipulation forces acting upon packets may be programmable so that packets may be manipulated along arbitrarily chosen or predetermined paths. An impedance sensor or other sensor may also be coupled to the PFP. The sensor may also be coupled to a controller which is coupled to the PFP. The impedance sensor, or other type of sensor, may be used to track the individual positions of packets so that it may be ensured that they are traveling along the correct path. Further, the positional information from the position sensor may aid various aspects of the fluidic analysis, as will be appreciated by those having skill in the art.

The electrode array of the PFP contains individual elements which can be addressed with DC, pulsed, or low frequency AC electrical signals (typically, less than about 10 kHz). Electrophoretic forces may be used instead of, or in addition to, other manipulation forces such as dielectrophoresis. One method of switching the voltages to the PFP is a CMOS high voltage chip. Another method uses a discrete switching network for injecting and moving droplets on passivated gold-on-glass PFP arrays.

The PFP may be used to manipulate packets or droplets of sample and reagents and can be used to overcome many difficulties found when using microfluidic valves and other system components. Microfluidic valves tend to be complex and leaky, the mixing of fluids at the ultra-low Reynold's numbers characteristic of small chambers is difficult, microfluidic metering is complicated, and all channel-based designs for these systems have reagent carryover and dead-space issues. Because droplets are discrete and can be efficiently injected with no moving parts under dielectrophoretic control, the quantized metering of samples and reagents may be readily accomplished. Droplets can be moved along arbitrarily chosen and crossing paths by DEP on a two dimensional reaction surface, eliminating the need for tubes and the vials required in channel-based fluidic designs. Furthermore, the ability to move droplets along arbitrary, crossing paths allows for full-programmability, and for multiplexed, parallel, and interleaved protocols to be readily executed.

b. Valves, Pumps, Injector, Reagent Metering and Routing

In one embodiment, droplet injection is a “valving” and “metering” action in which definite volumes of fluid are introduced from a pressurized reservoir (e.g. 2 to 10 psi) by electrically-gated dielectrophoretic forces. The injected droplets carry an intrinsic pressure, stored in the form of surface energy, and this not only induces spontaneous fusion of droplets when they are brought together but also is transferred when a droplet fuses with other fluid allowing, for example, the actuation of fluid flow in a channel. The PFP can be used for switching and metering droplets from several reservoirs and routing them to a reaction accumulator and regions where rinsing is needed. This is an ultra low-power, no moving parts, microscale method to accomplish completely programmable valving, metering and routing, and through the use of pre-pressurized reservoirs, it effectively eliminates the need for pumps.

A programmable fluid processor (PFP) can be configured to act as a programmable manifold that controls the dispensing and routing of all reagents. As used herein, a “program manifold” is meant to describe the combination of computer controlled forces and systems which are used to control the movement of fluids and packets through a biochip. The computer controlled forces are, for example, electric forces or magnetic forces. The movements of fluids and packets may be used, for instance, to move fluids or packets within a biochip, move fluids or packets into or out of the biochip, initiate or propagate a reaction, separate different components or other function, etc.

Electrode pads can be passivated and coated with anti-wetting agent such as TEFLON so that the routed droplets glide over the reaction surface. In one example, square electrode pads of 30 and 100 μm on a side were used to easily move droplets from less than one 1 to 6 pad widths; multiple pads can be energized to move larger droplets. The inventors have observed droplets moving at 15 to 4000 μm/sec depending on the DEP field. If two droplets are brought together, they will spontaneously fuse making combining their contents easy.

An injector can be used to inject droplets into a biochip. The static pressure differential necessary to maintain a droplet is expressed by

Pᵢₙ−Pₑₓₜ=γ/r

where Pᵢₙ and Pₑₓₜ are the internal and external hydrostatic pressures, γ the surface tension and the r the radius of the droplet. Thus, the pressure differential necessary to maintain a droplet is inversely proportional to the radius of the droplet. Since water adheres to hydrophilic glass, injected droplets tend to remain attached to the tip of the injector pipettes unless the outer surface is made hydrophobic. This can be done by dip-coating the pipettes in an anti-wetting agent such as Sigmacote®, a silicone solution in heptane, or a fluoropolymer, such as PFC1601A from Cytonis, Inc. Similar polar-nonpolar relationships can be used for the solvent systems for oligonucleotide synthesis and determine appropriate injector orifices and field strengths for OSE operations.

The injection of picoliter scale reagent aliquots are desired for the accurate metering and titration of reagent concentrations. For oligo synthesis, constant nanoliter-scale droplet size and constant nanomolar concentrations are desirable, which is readily attainable by adapting existing technology to the invention as described herein.

c. DEP Forces in Fluidic Systems

For a material of high dielectric constant Ed in a medium of lower dielectric constant εₘ, the time averaged DEP force in response to an alternating, inhomogeneous electrical field E based on the dipole approximation is given by

<F_{DEP}>=2πεₘr³Re

where r is the radius of the material. This force can be used to pull polar liquid droplets into a non-polar suspending phase and to attract droplets to high field regions on a switchable PFP electrode array.

Particles may be fabricated with a dielectric constant that is smaller than the suspending medium at certain frequencies and larger than it at others. Because the magnitude and direction of the DEP force are determined by the relationship between the medium and the particle dielectric constants, εₘ¹⁰⁸ and ε_{d}¹⁰⁸ particles may be subjected to attractive or repulsive DEP forces on demand by applying an electric field of appropriate frequency. These principles form one basis for the design of dielectrically-engineered beads.

Another useful characteristic of dielectrically-engineered beads is that, in an electrical field traveling in the x-direction, they experience a lateral traveling wave dependence of the phase of the field. Within an appropriate band of frequencies, this lateral TWD force may be used to transport a population of beads en masse within a suspending medium, and this may form the basis for actuation of metered delivery-on-demand for dielectric beads.

### V. OLIGONUCLEOTIDES

Oligonucleotides synthesized by methods of the current disclosure may be subjected to procedures before or after proofreading and error deletion. These procedures include hybridization, amplification, separation using chromatography or other techniques, and detection using, for example, impedance measurements or analysis using an indicator, mass spectroscopy, or other methods. These procedures can be accomplished while still on the PFP, in a microfluidic subunit attached to the PFP, or after removal from the PFP.

a. Nucleic Acid Hybridization

In the proofreading and error deletion methods described herein, hybridization of a synthesized sense and antisense oligonucleotide is required. As used herein, “hybridization”, “hybridizes” or “capable of hybridizing” shall be understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It shall also be understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

The nucleic acid segments of the present disclosure may be combined with other DNA sequences to produce a longer segment and may be combined with promoters, enhancers, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and the intended use.

In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

In general, it is envisioned that the hybridization probes as known in the art and described herein will be useful as reagents in hybridization. The selected conditions and probes used will depend on the particular circumstances based on the particular criteria required (depending, for example, on the G+C content, type of target nucleic acid, source of nucleic acid, size of hybridization probe, etc.).

b. Nucleic Acid Ampliflcation

The oligonucleotides synthesized with the present disclosure may undergo amplification, either on the PFP or after removal from the processor. Pairs of primers that selectively hybridize to nucleic acids may be contacted with the isolated nucleic acid under conditions that permit selective hybridization. The term “primer”, as defined herein, encompasses any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Once hybridized, the nucleic acid:primer complex may be contacted with one or more enzymes that facilitate template-dependent nucleic acid synthesis. Multiple rounds of amplification, also referred to as “cycles,” may be conducted until a sufficient amount of amplification product is produced. Next, the amplification product can be detected. In certain applications, the detection may involve determining impedance changes. Alternatively, the detection may involve visual detection or indirect identification of the product via chemiluminescence, radioactive scintigraphy of incorporated radiolabel or fluorescent label or even via a system using electrical or thermal impulse signals (Affymax technology).

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, each of which is incorporated herein by reference in entirety.

Other methods for amplification of the oligonucleotides include: ligase chain reaction (“LCR”), disclosed in EPA No. 320 308, Qbeta Replicase, described in PCT Application No. PCT/US87/00880, Strand Displacement Amplification (SDA), Repair Chain Reaction (RCR), using “modified” primers in a PCR-like, template- and enzyme-dependent synthesis as described in GB Application No. 2 202 328, using an excess of labeled probes where probe binds and is cleaved catalytically as described in PCT Application No. PCT/US89/01025, transcription-based amplification systems (TAS), including nucleic acid sequence based amplification (NASBA) and 3SR (Gingeras et al, PCT Application WO 88/10315), a nucleic acid amplification process involving cyclically synthesizing single-stranded RNA (“ssRNA”), ssDNA, and double-stranded DNA (dsDNA) as described by Davey et al, EPA No. 329 822, a nucleic acid sequence amplification scheme based on the hybridization of a promoter/primer sequence to a target single-stranded DNA (“ssDNA”) followed by transcription of many RNA copies of the sequence as described by Miller et al., PCT Application WO 89/06700, “RACE” and “one-sided PCR” (Frohman, 1990), and methods based on ligation of two (or more) oligonucleotides in the presence of nucleic acid having the sequence of the resulting “di-oligonucleotide”, thereby amplifying the di-oligonucleotide, may also be used in the amplification step of the present disclosure. Each reference mentioned in this paragraph is hereby incorporated by reference.

One example of chip based amplification is, PCT Application No. WO 94/05414, to Northrup and White, incorporated herein by reference, which reports an integrated micro-PCR™ apparatus for collection and amplification of nucleic acids from a specimen.

c. Nucleic Acid Detection

In certain embodiments, it may be advantageous to employ an appropriate means to determine oligonucleotide position and/or hybridization. The oligonucleotide may be detected using impedance measurements. Similarly, a wide variety of appropriate indicator means are known in the art, including fluorescent, radioactive, enzymatic or other ligands, such as avidin/biotin, which are capable of being detected. Fluorescent labels or an enzyme tags such as urease, alkaline phosphatase or peroxidase may be used instead of radioactive or other environmentally undesirable reagents. In the case of enzyme tags, calorimetric indicator substrates are known that can be employed to provide a detection means visible to the human eye or spectrophotometrically, to identify specific hybridization with complementary nucleic acid-containing samples.

In one embodiment, visualization may be used to study the oligonucleotide. A typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation. Visualization may be achieved indirectly. Following separation of amplification products, a labeled, nucleic acid probe may be brought into contact with the amplified marker sequence. The probe preferably may be conjugated to a chromophore but may be radiolabeled. In another embodiment, the probe may be conjugated to a binding partner, such as an antibody or biotin, and the other member of the binding pair carries a detectable moiety.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out aspects of methods according to the present invention. Other examples, U.S. Pat. Nos. 5,304,487 to Wilding et al., and 5,296,375 to Kricka et al., each of which is incorporated herein by reference, discuss devices for collection and analysis of cell containing samples and are both incorporated herein by reference. U.S. Pat. No. 5,856,174, which is incorporated herein by reference, describes an apparatus which combines the various processing and analytical operations involved in nucleic acid analysis.

d. Chromatographic Techniques

Separation of proofread oligonucleotides from a reaction mixture may be done by holding the oligonucleotide attached to a solid support by a DEP induced force or another force while flowing a solution through the chamber to remove all material that is not bound to the beads. It may also be desirable to separate the oligonucleotides from beads or from other components in a reaction chamber; separation of samples that are obtained to interact with the synthesized oligonucleotides may also be done.

Samples may be separated by agarose, agarose-acrylamide or polyacrylamide gel electrophoresis using standard methods (Sambrook et al., 1989, which is incorporated herein by reference). Alternatively, chromatographic techniques may be employed to effect separation. There are many kinds of chromatography which may be used in conjunction with the present disclosure: adsorption, partition, ion-exchange and molecular sieve, and many specialized techniques for using them including column, paper, thin-layer and gas chromatography (Freifelder, 1982). In yet another alternative, labeled oligonucleotide produces, such as biotin-labeled or antigen-labeled can be captured with beads bearing avidin or antibody, respectively.

Microfluidic techniques include separation on a platform such as microcapillaries, designed by ACLARA BioSciences Inc., or the LabChip™ “liquid integrated circuits” made by Caliper Technologies Inc. The automated separation of oligonucleotides in a microfluidic environment has been described by Chandler et al. (2000) and Bruckner-Lea et al. (2000).

e. Mass Spectroscopy

Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). For low molecular weight molecules, mass spectrometry has been part of the routine physical-organic repertoire for analysis and characterization of organic molecules by the determination of the mass of the parent molecular ion. In addition, by arranging collisions of this parent molecular ion with other particles (e.g., argon atoms), the molecular ion is fragmented forming secondary ions by the so-called collision induced dissociation (CID). The fragmentation pattern/pathway very often allows the derivation of detailed structural information. Other applications of mass spectrometric methods known in the art can be found summarized in Methods in Enzymology, Vol. 193: “Mass Spectrometry” (J. A. McCloskey, editor), 1990, Academic Press, New York, which is incorporated herein by reference.

Due to the apparent analytical advantages of mass spectrometry in providing high detection sensitivity, accuracy of mass measurements, detailed structural information by CID in conjunction with an MS/MS configuration and speed, as well as on-line data transfer to a computer, there has been considerable interest in the use of mass spectrometry for the structural analysis of nucleic acids. Reviews summarizing this field include K. H. Schram (1990); and P. F. Crain (1990). The biggest hurdle to applying mass spectrometry to nucleic acids is the difficulty of volatilizing these very polar biopolymers. Therefore, “sequencing” had been limited to low molecular weight synthetic oligonucleotides by determining the mass of the parent molecular ion and through this, confirming the already known sequence, or alternatively, confirming the known sequence through the generation of secondary ions (fragment ions) via CID in an MS/MS configuration utilizing, in particular, for the ionization and volatilization, the method of fast atomic bombardment (FAB mass spectrometry) or plasma desorption (PD mass spectrometry). As an example, the application of FAB to the analysis of protected dimeric blocks for chemical synthesis of oligodeoxynucleotides has been described (Koster et al. 1987).

Two ionization/desorption techniques are electrospray/ionspray (ES) and matrix-assisted laser desorption/ionization (MALDI). ES mass spectrometry was introduced by Fenn et al. 1984; WO 90/14148 and its applications are summarized in review articles (R. D. Smith et al. 1990; B. Ardrey, 1992). As a mass analyzer, a quadrupole is most frequently used. The determination of molecular weights in femtomole amounts of sample is very accurate due to the presence of multiple ion peaks which all could be used for the mass calculation.

MALDI mass spectrometry, in contrast, can be particularly attractive when a time-of-flight (TOF) configuration is used as a mass analyzer. The MALDI-TOF mass spectrometry has been introduced by Hillenkamp et al. (1990). Since, in most cases, no multiple molecular ion peaks are produced with this technique, the mass spectra, in principle, look simpler compared to ES mass spectrometry. DNA molecules up to a molecular weight of 410,000 Daltons may be desorbed and volatilized (Williams et al., 1989). More recently, the use of infra red lasers (IR) in this technique (as opposed to UV-lasers) has been shown to provide mass spectra of larger nucleic acids such as, synthetic DNA, restriction enzyme fragments of plasmid DNA, and RNA transcripts up to a size of 2180 nucleotides (Berkenkamp et al., 1998). Berkenkamp et al., 1998, also describe how DNA and RNA samples can be analyzed by limited sample purification using MALDI-TOF IR.

In Japanese Patent 59-131909, which is incorporated herein by reference, an instrument is described which detects nucleic acid fragments separated either by electrophoresis, liquid chromatography or high speed gel filtration. Mass spectrometric detection is achieved by incorporating into the nucleic acids atoms which normally do not occur in DNA such as S, Br, I or Ag, Au, Pt, Os, Hg.

### VII. EXAMPLES

The following examples are included to demonstrate specific embodiments of the invention. It should be appreciated by those of skill in the art that the techniques disclosed in the examples which follow represent techniques discovered by the inventors to function well in the practice of the invention, and thus can be considered to constitute specific, non-limiting modes for its practice. However, those of skill in the art should, in light of the present disclosure, appreciate that many changes can be made in the specific embodiments which are disclosed and still obtain a like or similar result without departing from the spirit and scope of the inventions defined by the claims.

### Example 1

To initiate an oligonucleotide synthesis cycle in a PFP, OH derivatized microbead support are dispensed from a solid phase reservoir by TWD and carried down a fluid channel by droplets delivered upstream by the PFP. As the beads enter the accumulator, an interdigitated, dielectrophoretic electrode is used to trap the beads by positive DEP. Beads are immobilized, and the reaction solution is injected into the PFP and programmably routed to the accumulator where the support beads are located. The beads are then sequentially perfused with the required sequence of nucleoside/nucleotide monomers, coupling, deprotection, and other necessary chemistries as understood to be used in phosphoramidite synthesis to produce the desired oligonucleotide.

Once synthesis is complete, beads are released by negative DEP and flushed from the PFP, carrying the proofread DNA on their surfaces. The next bead dispensing and custom synthesis cycle may then be initiated.

After synthesis, oligonucleotides can be deprotected and cleaved from the beads and analyzed using capillary reverse phase HPLC and MALDI-TOF. Thus synthesis reactions can be evaluated rapidly allowing reaction conditions to be readily optimized.

### Example 2

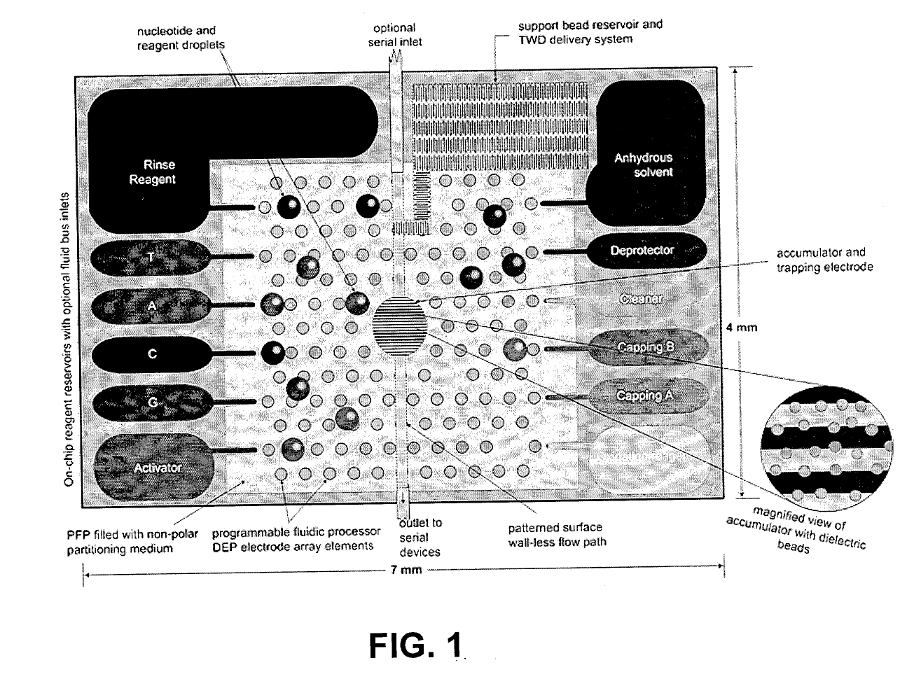

A schematic drawing of a 4 mm×7 mm unit cell module is shown in FIG. 1. The left- and right-most sections contain on-chip reagent reservoirs that may be optionally interfaced to a fluidic bus. The central portion includes a programmable fluidic processor (PFP) that may use dielectrophoresis (DEP) to inject small (e.g., 5 nL) droplets of reagents on demand from the reservoirs into the PFP reaction space where they are routed along arbitrarily-programmable paths defined by DEP forces provided by a two-dimensional array of electrodes. The reaction space may be filled with a low-dielectric constant, immiscible partitioning fluid medium such as decane or bromodoecane. The DEP injection may provide all fluid metering and valving actions required for synthesis including flushing completed oligonucleotides from the synthesizer. The electrode array may be passivated with an inert coating (e.g., TEFLON) to eliminate the possibility of surface contamination or contact of reagents with the metal electrodes. In order to further obviate chemical interactions with device surfaces, oligonucleotides may be synthesized on the surfaces of mobile, solid phase supports developed for this purpose rather than on a device itself. These supports may be 10 micron beads (although other sizes may be used with the same, or similar, results) engineered so as to give them well-defined dielectric properties that permit them to be tapped and released by DEP as required. The bead supports may be stored in an on-chip reservoir (top right of the center channel) and metered and dispensed on demand by traveling wave dielectrophoresis (TWD) provided by a four-phase TWD electrode track on the bottom surface of the reservoir. As it will be understood by those having skill in the art with the benefit of this disclosure, other electronic and/or mechanically-induced forces may be used to manipulate, meter, and dispense supports.

### Example 3

In the drawing of FIG. 1, the accumulator volume is 12 nL and droplet sizes may be 250 microns/12 nL. The support beads may be about 10 microns in diameter, providing a surface area of about 3×10⁻¹⁰ m² and, at 1% surface coverage, a capacity of 10⁻¹ oligos per bead. At a charge for the accumulator of 1000 beads, this provides support for 10⁷ oligos in each synthesis run. Each small reservoir shown in FIG. 1 holds enough reagent for 160 dispensing droplets and the bead reservoir holds enough beads for 100 synthesis runs. For typical oligo synthesis, larger reservoirs, an external reagent tank analogous to those used in ink jet printheads, or a fluid bus for off-chip delivery of reagents may be used.

All of the techniques disclosed and claimed herein can be made and executed without undue experimentation in light of the present disclosure. While the compositions and methods of this invention have been described in terms of specific embodiments, it will be apparent to those of skill in the art that variations may be applied to the apparatuses and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. Further, it will be apparent that a wide variety of manipulation forces other than dielectrophoresis may be used such as electrophoresis, mechanical, and/or optical forces, just to name a few. All such similar substitutes and modifications apparent to those skilled in the art are deemed to be within the spirit, scope and concept of the invention as defined by the appended claims.

### REFERENCES

Each of the following references is specifically incorporated herein by reference:

Axton et al., Journal of Medical Genetics 1997 Apr;34(4):279-86.

Barany, F. Proc. Natl. Acad. Sci 88: 189-193, 1991.

Beaucage, and Lyer, Tetrahedron, 48:2223-2311, 1992.

Bonfield J K, Rada C, Staden R. Automated detection of point mutations using fluorescent sequence trace subtraction. Nucleic Acids Research 1998;26:3404-9.

Bruckner-Lea C J, Stottlemyre M S, Holman D A, Grate J W, Brockman F J, Chandler D P. “Rotating rod renewable microcolumns for automated, solid-phase DNA hybridization studies.” Anal Chem. September 1;72(17):4135-41, 2000.

Chandler D P, Holman D A, Brockman F J, Grate J W, and Bruckner-Lea C J, “Renewable Microcolumns for Solid Phase Nucleic Acid Separations and Analysis from Environmental Samples,” TrAC, Trends Anal. Chem., 9, 314-321 (2000).

Chow C S, Barton J K. “Recognition of G-U mismatches by tris(4,7-diphenyl-1,10-phenanthroline)rhodium(III).” Biochemistry Jun. 23, 1992;31(24):5423-9.

Cotton R G H. Current methods of mutation detection. Mutation Research 1993;285:125-44.

Cotton, Rodrigues, Campbell, “Reactivity of cytosine and thymine in single-base-pair mismatches with hydoxylamine and osmium tetroxide and its application to the study of mutations,” Proc. Natl. Acad. Sci. USA, 85(12):4397-4401, 1988.

Crain P F, McCloskey J A. “Applications of mass spectrometry to the characterization of oligonucleotides and nucleic acids.” Curr Opin Biotechnol. February 1998;9(1):25-34.

Deeble V J, Roberts E, Robinson M D, Woods C G, Bishop D T, Taylor G R. Comparison of Enzymne Mismatch Cleavage and Chemical Cleavage of Mismatch on a Defined Set of Heteroduplexes. Genetic Testing 1998 in press.

Draghia et al., Human Mutation 1997;9(3):234-42.

Ekins R, Chu F W. “Microarrays: their origins and applications.” Trends Biotechnol. June 1999; 17(6):217-8.

Ellis L A, Taylor G R, Banks R, Baumberg S. MutS binding protects heteroduplex DNA from exonuclease digestion in vitro—a simple method for detecting mutations. Nucleic Acids Research 1994;22:2710-1.

Ellis T P, Humphrey K E, Smith M J, Cotton R G., Hum Mutat 1998;11(5):345-53.

Fishel R, Wilson T. MutS homologs in mammalian cells. Current Opinion in Genetics & Development 1997;7:105-13.

Gao, Yu, LeProust, Sonigo, Pellois, Zhang, “Oligonucleotide synthesis using solution photogenerated acids,” J. Am. Chem. Soc., 120:12698-12699, 1998.

Gascoyne, Wang, Huang, Becker, “Dielectrophoretic separation of cancer cells from blood,” IEEE Transactions on Industry Applications, 33(3):670-678, 1997.

Germain et al. Human Genetics 1996 Dec; 98(6):719-26.

Gibbons R J, Bachoo S, Picketts D J, Higgs D R. Identification of mutations in ATRX by RNase mismatch cleavage. American Journal of Human Genetics 1997;61:1955-.

Giddings J C, Unified Separation Science, John Wiley & Sons, Inc, New York, N.Y. 1991.

Goldrick M M, Kimball G R, Liu Q, Martin L A, Sommer S S, Tseng J Y H. Nirca(Tm)—A Rapid Robust Method For Screening For Unknown Point Mutations. Biotechniques 1996;21:106-12.

Gordon, L. K., and Haseltine, W. A.: Comparison of the cleavage of pyrimidine dimers by the bacteriophage T4 and Micrococcus luteus, Journ. Biol. Chem., 255, 12047 (1980).

Grange D K, Gottesman G S, Lewis M B, Marini J C. Detection Of Point Mutations In Type-I Collagen By RNase Digestion Of RNA RNA Hybrids. Nucleic Acids Research 1990;18:4227-36.

Hawkins G A, Hoffman L M. Accurate and efficient mutation detection and localization using base excision sequence scanning (BESS). American Journal of Human Genetics 1997;61:1279.

Hsu I C, Yang Q P, Kahng M W, Xu J F. Detection of DNA point mutations with DNA mismatch repair enzymes. Carcinogenesis 1994; 15:1657-62.

Jackson and Barton, “Recognition of DNA base mismatches by a rhodium intercalator,” J. Am. Chem. Soc., 119:12986-12987, 1997.

Jackson B A, Alekseyev V Y, Barton J K “A versatile mismatch recognition agent: specific cleavage of a plasmid DNA at a single base mispair.” Biochemistry Apr. 13, 1999 13;38(15):4655-62.

Jackson, Alekseyev, Barton, “A versatile mismatch recognition agent: specific cleavage of a plasmid DNA at a single base mispair,” Biochemistry, 38(15):4655-4662, 1999.

Katzhendler, Cohen, Rahamim, Weisz, Ringel, Deutsch, “The effect of spacer, linkage and solid support on the synthesis of oligonucleotides,” Tetrahedron, 45:2777-2792, 1989.

Kisko J L, Barton J K., “Recognition of DNA base pair mismatches by a cyclometalated Rh(III) intercalator” Inorg Chem Oct 16, 2000;39(21):4942-9.

Laboratory Methods for the Detection of Mutations and Polymorphisms in DNA CRC Press 1997 Ed GR Taylor.

Lahue R S, Au K G, Modrich P. DNA mismatch correction in a defined system. Science 1989;245: 160-4.

Lambrinakos, Humphrey, babon, Ellis, Cotton, “Reactivity of potassium permanganate and tetraethylammonium chloride with mismatched bases and a simple mutation detection protocol,” Nuc. Acids Res., 27(8):1866-1874, 1999.

LeProust, Pellois, Yu, Zhang, Srivannavit, Gulari, Zhou, Gao, “Combinatorial screening method for synthesis optimization on a digital light-controlled microarray platform,” J. Comb. Chem., 2:355-360, 2000.

LeProust, Zhang, Yu, Gao, Xiang, Zhou, “Characterization of oligodeoxyribonucleotide synthesis on glass plates,” Nucleic Acids Res., 2001.

Lloyd, R. S., Hanawalt, P. C., and Dodson, M. L.: Processive action of T4 endonuclease V in UV-irradiated DNA, Nucleic Acids Res., 183, 109 (1980).

Lu A L, Hsu I C. Detection of single DNA-base mutations with mismatch repair enzymes. Genomics 1992;14:249-55.

Mashal R D, Koontz J, Sklar J. Detection of mutations by cleavage of DNA heteroduplexes with bacteriophage resolvases. Nature Genetics 1995;9:177-83.

Meyer and Day, BioTechniques 30:584-593, 2001.

Modrich P. Strand-specific mismatch repair in mammalian cells. Journal of Biological Chemistry 1997;272:24727-30.

Myers R M, Larin Z, Maniatis T. Detection Of Single Base Substitutions By Ribonuclease Cleavage At Mismatches In RNA-DNA Duplexes. Science 1985;230:1242-6.

Neddermann P, Gallinari P, Lettieri T, Schmid D, Truong O, Hsuan J J, Wiebauer K, Jiricny J. Cloning and expression of human G/T mismatch-specific thymine-DNA glycosylase. Journal of Biological Chemistry 1996;271:12767-74.

Oleykowski C A, Bronson-Mullins C R, Godwin A K, Yeung A T. Mutation detection using a novel plant endonuclease. Nucleic Acids Research 1988;16:4597-602.

Parsons B L, Heflich R H. Evaluation of MutS as a tool for direct measurement of point mutations in genomic DNA. Mutation Research-Fundemental and Molecular Mechanisms of Mutagenesis 1997;374:277-85.

Pellois, Wang, Gao, “Peptide synthesis based on t-Boc chemistry and solution photogenerated acids,” J. Comb. Chem., 2:349-354, 2000.

Pohler J G, Giraudpanis M E, Lilley D J. T4 endonuclease VII selects and alters the structure of the four-way DNA junction; Binding of a resolution-defective mutant enzyme. Journal of Molecular Biology 1996;260:678-96.

Roberts, Deeble, Woods, Taylor, “Potassium permanganate and tetraethylammonium chloride are a safe and effective substitute osmium tetroxide in solid-phase fluorescent chemical cleavage of mismatch,” Nuc. Acids Res., 25(16):3377-3378, 1997.

Rossiter B F, Caskey C T. Molecular scanning methods of mutation detection. Journal of Biological Chemistry 1990;265:12753-6.

Rowley G, Saad S, Gianelli F, Green P M. Ultrarapid mutation detection by multiplex, solid-phase chemical cleavage. Genomics 1995;30:574-82.

Saleeba, et al., Methods Enzymology, 217:286-295, 1993.

Smith J, Modrich P. Mutation detection with MutH, MutL, and MutS mismatch repair proteins. Proceedings Of The National Academy Of Sciences Of The United States Of America 1996;93:4374-9.

Smith J, Modrich P. Removal of polymerase-produced mutant sequences from PCR products. Proc Natl Acad Sci (US) 1997;94:6847-50.

Taylor G R “Enzymatic and Chemical Cleavage Methods” http://www.leeds.ac.uk/cmgs/-leedsdna/science/cleavage/review.htm.

Taylor G R, Deeble J., “Enzymatic methods for mutation scanning.” Genet Anal 1999 Feb; 14(5-6):181-6.

Wang, Huang, Gascoyne, Becker, “Dielectrophoretic manipulation of particles,” IEEE Industrial App. Soc., 33(3):660-669, 1997.

Wang, Huang, Vykoukal, Becker, Gascoyne, “Cell separation by dielectrophoretic field-flow-fractionation, ” Analytical Chemistry, 72(4):832-839, 2000.

Wang, Pethig, Jones, “Relationship of dielectrophoretic and electrorotational behavior exhibited by polarized particles,” J. Phys. D Appl. Phys., 25:905-912, 1992.

Wasserman, Tao, Whitesides, “Structure and reactivity of alkylsiloxane monolayers formed by reaction of alkytrichlorosilanes on silicon substrates,” Langmuir, 5:1074-1087, 1989.

White, Giraud-Panis, Pohler, Lilley, “Recognition and manipulation of branched DNA structure by junction-resolving enzymes,” J. Mol. Biol., 269:647-664, 1997.

Wu, D. Y. and R. B. Wallace Gene, 76:245-254, 1989.

Yao and Kow, “Further characterization of E. coli endonuclease V” mechanism of recognition for deoxyinosine, deoxyuridine, and base mismatches in DNA,“J. Biol. Chem., 272(49):30774-30779, 1997.

Youil et al., Hum Mutat 2000 Jul;16(1):92-3.

Youil R, Kemper B, Cotton RGH. Detection of 81 of 81 known mouse beta-globin promoter mutations with T4 Endonuclease-VII-The EMC Method. Genomics 1996;32:431-5.

Youil, Kemper, Cotton, “Screening for mutations by enzyme mismatch cleavage with T4 endonuclease VII,” Proc. Natl. Acad. Sci. USA, 92(1):87-91, 1995.

What is claimed is:

1. A method of solid-phase oligonucleotide synthesis comprising:
synthesizing a sense oligonucleotide;
synthesizing an antisense oligonucleotide;
annealing said sense and antisense oligonucleotides to form double stranded DNA (dsDNA);
capping the ends of said dsDNA;
cleaving said dsDNA wherein cleavage occurs at or near a Watson-Crick base pair mismatch; and
digesting uncapped dsDNA.

2. The method of claim 1, further comprising digesting one strand of said dsDNA.

3. The method of claim 1, wherein said oligonucleotide contains 5-100 bases.

4. The method of claim 1, wherein the method occurs on a biochip.

5. The method of claim 4, further comprising using the synthesized oligonucleotide without removing the oligonucleotide from said biochip.

6. The method of claim 1, wherein said solid-phase comprises beads.

7. The method of claim 6, wherein said beads are 2-50 μm in diameter.

8. The method of claim 6, wherein said beads comprise dielectrically-engineered beads that are manipulated by dielectrophoresis.

9. The method of claim 6, wherein said beads are gold coated polystyrene beads.

10. The method of claim 6, wherein said beads are coated with a phospholipid.

11. The method of claim 6, wherein said beads are coated with a polyethylene glycol.

12. The method of claim 1, wherein an enzyme is used to cleave said dsDNA.

13. The method of claim 12, wherein said enzyme is an E. Coli endonuclease.

14. The method of claim 12, wherein said enzyme is T7 endonuclease I.

15. The method of claim 1, wherein said dsDNA is cleaved chemically.

16. The method of claim 15, wherein potassium permanganate and hydroxylamine are used to cleave said dsDNA.

17. The method of claim 15, wherein a photoactivated rhodium DNA intercalator is used to cleave said dsDNA.

18. The method of claim 1, wherein a combination of enzymes and/or chemicals are used to cleave said dsDNA.

19. The method of claim 1, further comprising analysis of the DNA with MALDI-TOF MS.

20. The method of claim 1, further comprising using laser assisted deprotection.

21. The method of claim 1, further comprising activating proofreading using laser assisted proofreading activation.

22. The method of claim 1, further comprising control software for the injection and manipulation of fluid droplets on a programmable fluid processor.

23. The method of claim 22, wherein said programmable fluid processor is used for reagent routing and delivery.

24. An apparatus for performing the method of claim 1.

25. A method of forming long polynucleotides comprising:
synthesizing a first proofread double stranded DNA (dsDNA) wherein the synthesis comprises:
synthesizing a sense oligonucleotide;
synthesizing an antisense oligonucleotide;
annealing said sense and antisense oligonucleotides to form dsDNA;
capping the ends of said dsDNA;
cleaving said dsDNA wherein cleaved dsDNA occurs at or near a Watson-Crick base pair mismatch; and
digesting uncapped dsDNA;
synthesizing a second proofread dsDNA; and
ligating said first proofread DNA with said second proofread DNA to form a long polynucleotide.

26. The method of claim 25, wherein 2 -2000 proofread dsDNA are ligated to form said long polynucleotide.

27. The method of claim 26, wherein 10 -500 proofread dsDNA are ligated to form said long polynucleotide.

28. The method of claim 25, wherein the proofread dsDNA are synthesized in parallel.

29. The method of claim 25, wherein the proofread dsDNA are synthesized sequentially.

30. The method of claim 25, wherein ligation occurs using a T4 ligase.

31. The method of claim 25, further comprising digesting one strand of said dsDNA.

32. The method of claim 25, wherein said synthesis and ligation occur on a biochip.

33. The method of claim 32, further comprising a programmable fluidic processor.

34. The method of claim 33, wherein said programmable fluidic processor is used for reagent routing and delivery.

35. The method of claim 31, further comprising using the synthesized oligonucleotide without removing the oligonucleotide from said biochip.

36. An apparatus for performing the method of claim 25.
